(19) United States
(12) Patent Application Publication
(10) Pub. No.: US 2006/0253068 A1
(43) Pub. Date: Nov. 9, 2006

(54) Use of biocompatible in-situ matrices for delivery of therapeutic cells to the heart

(76) Inventors: Paul van Bilsen, Maastricht (NL); Edze Tijsma, Maastricht (NL)

Correspondence Address: DIEHL SERVILLA LLC, 77 BRANT AVE, SUITE 110, CLARK, NJ, 07066, US

(21) Appl. No.: 11/110,415

(22) Filed: Apr. 20, 2005

(51) Int. Cl. A61M 31/00; A61M 37/00

(52) U.S. Cl. 604/93.01; 604/522

(57) ABSTRACT

The present invention provides novel methods and systems for delivering therapeutic cells to the heart of a subject.

### FIELD OF THE INVENTION

The present invention relates generally to the use of biocompatible matrices for the delivery of therapeutic agents, such as cells and growth factors, to the heart.

### BACKGROUND OF THE INVENTION

There is an increasing number of patients with coronary heart disease that cannot be treated either by bypass grafting or interventional coronary artery procedures despite the fact that viable myocardium has been demonstrated by techniques like PET or stress echocardiography. Recently, transmyocardial revascularization (TMR), in which laser energy is applied to create intramyocardial channels in order to restore blood supply to ischemic areas and to stimulate neoangiogenesis, was shown to lower angina scores, increase exercise tolerance time, and improve perception of quality of life in patients with refractory angina pectoris (Burkhoff et al., Lancet, 354:885 (1999)).

In addition, the feasibility and short-term effects of creating intramyocardial channels by means of high frequency (HF) current ablation with heat denaturation have been investigated in an in vivo rabbit model (Dietz et al., Cardiology, 93:234 (2000)). Intramyocardial channels were created, the majority of which remained patent for at least a small amount of time. Others, however, have found different stages of wound healing in human nonresponder myocardium after TMR, resulting in scarred tissue that displayed capillary networks and dilated venules without evidence of patent and endothelialized laser-created channels (e.g., Gassler et al., Circulation, 95:371 (1997)).

Vascularization of intramyocardial channels could potentially be improved through the use of angiogenic growth factors. Therapeutic benefit has been demonstrated following bolus injection or systemic administration of growth factor (see, e.g., Takeshita et al., J. Clin. Invest. 93:662 (1994); Hendel et al., Circulation 101:118 (2000)). This strategy is limited, however, by the inherent instability of many proteins in vivo and the potential for uncontrolled activities at undesired sites (Simons et al., Circulation 102:E73 (2000)).

Intramyocardial channel treatment could also be improved through the induction of cardiomyocyte proliferation. However, since cardiomyocytes lack the ability to regenerate, cardiac damage resulting from cardiac cell death is permanent. Several approaches involving myocyte transplantation are currently under investigation to repair damaged cardiac tissue, including transplantation of cells from allogeneic, xenogeneic, transgeneic, and autogeneic sources (see Oakley et al., Ann. Thorac. Surg. 71:1724 (2001)). As with growth factor administration, myocyte transplantation suffers from the potential for uncontrolled activities at undesired sites (Simons et al., Circulation 102:E73 (2000)).

One approach to retaining therapeutic agents in intramyocardial channels, such as those formed by TMR, is the through use of a thixotropic agent. For example, Yamamoto et al. (Basic Res. Cardiol. 95:55 (2000)), discloses the use of a thixotropic gel for administration of bFGF to enhance the angiogenic effects of TMR. Similarly, U.S. Pat. No. 6,524,298 discloses the use of a thixotropic gel to retain various growth factors and gene therapy vectors in intramyocardial channels. The success of thixotropic agents, however, depends on each operator's ability to maintain proper viscosity during administration. Furthermore, the shear force necessary to maintain the thixotropic agent in the fluid state during administration can be harmful to living cells.

Another approach to retaining therapeutic agents in intramyocardial channels is disclosed in U.S. Patent Publication No. 2004/0009155, wherein cells (e.g., cardiomyocytes) are introduced into a target area via, e.g., TMR, and a plug, which may contain growth factors, is deposited at the introduction site. The plug member may be pre-formed or may form in-situ. Such plugs, however, may be subject to leakage and do not provide a suitable matrix for the growth and proliferation of the introduced cells. Another drawback to this approach is that the delivery of the therapeutic agent is not uniform throughout the intramyocardial channel. Similarly, U.S. Pat. No. 6,045,565 describes a variety of adhesives, including fibrin glue and cyanoacrylates, for retaining angiogenic material within intramyocardial channels, none of which are suitable for introducing therapeutic cells.

As a result, there is an immediate need for improved methods and systems for effectively delivering therapeutic cells to the heart of a subject.

### SUMMARY OF THE INVENTION

The present invention fills the foregoing need by providing novel methods and systems for delivering therapeutic agents to the heart of a subject. Applicants have found that delivery of therapeutic agents, such as cells and growth factors, to cardiac tissue using a biocompatible matrix that forms in situ upon application of an external stimulus improves the efficacy of intramyocardial channel treatment of cardiac tissue in a manner more uniform than delivery of a therapeutic agent from a plug. The use of such a matrix retains the therapeutic agent in the intramyocardial channel, as well as providing a suitable support for the growth and proliferation of therapeutic cells.

Accordingly, one aspect of the present invention is directed to a method for delivering therapeutic cells to the heart of a subject, comprising: a) forming one or more channels within a region of a wall of the subject's heart which includes a myocardial layer; and b) delivering to said region a composition comprising living cells and a biocompatible matrix that forms in situ upon exposure to a physiological condition, wherein said living cells provide a therapeutic effect. In some embodiments, the therapeutic cells are contractile cells. In other embodiments, the therapeutic cells secrete a growth factor. In some embodiments, the biocompatible matrix is a thermoplastic paste; an in situ crosslinked system, such as a thermoset, or an ion-mediated gelating system; an in situ precipitating system with a sol-gel transition induced, for example, by solvent removal, or by temperature or pH; or an organogel. In further embodiments, the composition further comprises one or more therapeutic agents.

Another aspect of the present invention is directed to a method for treating a patient suffering from heart disease, comprising: a) forming one or more channels within a region of a wall of the patient's heart which includes a myocardial layer; and b) delivering to said region a composition comprising living cells and a biocompatible matrix that forms in situ upon exposure to a physiological condition, wherein said living cells provide a therapeutic effect.

Another aspect of the present invention is directed to a system for delivering therapeutic cells to the heart of a subject, comprising: a) means for forming one or more channels within a region of a wall of the subject's heart which includes a myocardial layer; (b) means for introducing into said region a composition comprising living cells and a biocompatible matrix that forms in situ upon exposure to a physiological condition, wherein said living cells provide a therapeutic effect. In some embodiments, the channel forming means are provided by laser transmyocardial revascularization, high frequency current transmyocardial revascularization, percutaneous laser myocardial revascularization, high frequency current myocardial revascularization, mechanical transmyocardial revascularization or mechanical percutaneous myocardial revascularization. In some embodiments, the composition delivery means comprises a catheter and a delivery element such as a needle based injection system.

Another aspect of the present invention is directed to a system for delivering therapeutic cells to the heart of a patient suffering from heart disease, comprising: a) means for forming one or more channels within a region of a wall of the patient's heart which includes a myocardial layer; and (b) means for introducing into said region a composition comprising living cells and a biocompatible matrix that forms in situ upon exposure to a physiological condition, wherein said living cells provide a therapeutic effect.

### DETAILED DESCRIPTION OF THE INVENTION

The present invention relates to methods and systems for delivering therapeutic cells to the heart of a subject. As used herein, the term “subject” refers to a mammal that may benefit from the administration of a composition or method of this invention. Examples of subjects include humans, and other animals such as horses, pigs, cattle, dogs, cats, rabbits, and aquatic mammals.

Accordingly, a first aspect of the present invention is directed to a method for delivering therapeutic cells to the heart of a subject, comprising: a) forming one or more channels within a region of a wall of the subject's heart which includes a myocardial layer; and b) delivering to said region a composition comprising living cells and a biocompatible matrix that forms in situ upon exposure to a physiological condition, wherein said living cells provide a therapeutic effect.

As used herein, “a biocompatible matrix that forms in situ upon exposure to a physiological condition” means a non-toxic material, preferably biodegradable, that solidifies or semi-solidifies upon exposure to a physiological condition in vivo, such as, e.g., temperature, pH, water content and/or ion concentration. Such biocompatible matrices are well known in the art and include, e.g., thermoplastic pastes (i.e., matrices that form upon cooling), thermosets (i.e., matrices that form upon heating), ion-mediated gelating systems (i.e., matrices that form upon contact with divalent cations), temperature-, pH-, and solvent removal-induced sol-gels (i.e., matrices that form upon precipitation from solution), and organogels (i.e., matrices composed of water-insoluble amphiphilic lipids which swell in water) (Hatefi and Amsden, J. Control. Release 80:9 (2002)). Components useful for the preparation of these biocompatible matrices include, e.g., poly-D,L-lactide, poly-L-lactide, polyglycolide, poly-ε-caprolactone, polytrimethylene carbonate, polydioxanone, poly(ortho esters), polymers of glycerol esters of fatty acids, poly(acrylic acid), poly(methacrylic acid), poly(ethylene glycol), carbopol, hydroxypropylmethylcellulose, chitosan, poly(N-isopropyl acrylamide), dextran-(L)lactate, dextran-(D)lactate, block copolymers of poly(ethylene oxide) and poly(propylene oxide), and mixtures thereof. More specifically, thermoplastic pastes include materials that have a melting temperature above body temperature, preferably between 25° and 65° C., such as polymers or copolymers prepared from monomers such as D,L-lactide, glycolide, ε-caprolactone, trimethylene carbonate, dioxanone, ortho esters and poly(ethylene glycol), and blends of these (co)polymers. Ion-mediated gelating systems include alginate. Solvent-removal precipitating systems include sucrose acetate isobutyrate and water-insoluble polymers dissolved in water-miscible, physiologically compatible solvents, such as poly(lactide-co-glycolide) and poly(acrylic acid). Temperature-induced systems include polymers such as poly(N-isopropylacrylamide) (PNIPAAM), methylcellulose (MC), MC-grafted PNIPAAM, poly(ethylene glycol)-poly(lactic acid)-poly(ethyleneglycol) triblocks (PEG-PLA-PEG), PEG-PLA diblock copolymers, poly(ethylene oxide)-poly(propylene oxide)-poly(ethylene oxide) (PEO-PPO-PEO) triblocks (Pluronics® or Poloxamer®), capped PEO-PPO-PEO, PEO-poly(L-lactic acid-co-glycolic acid) (PEO-PLLGA), PEO-poly(DL-lactic acid-co-glycolic acid (PEO-PLGA) block and graft copolymers, PEG-PLGA-PEG, PLGA-PEG-PLGA, poly(organophosphazene)s, chitosan-based, and silk-elastin polymers. pH-induced systems include hydroxypropyl-cellulose (Carbopol®), chitosan and alginate. Organogels include oils such as peanut oil and waxes. Preferably, the polymers are modified to facilitate cell adhesion and cell growth. Such modifications include, but are not limited to, introduction of RGD-sites on the polymer chains.

The compositions of the present invention comprise therapeutic cells in contact with the biocompatible matrix. The cells can be pre-mixed with the matrix, or the cells and matrix can be delivered separately such that they contact in the intramyocardial channels. Cells compatible with the methods of the present invention include any cell capable of providing a therapeutic effect. The therapeutic effect can be structural, mechanical or biological, or combinations thereof. In some embodiments, the therapeutic cells are capable of forming new contractile tissue in and/or near the intramyocardial channels. The cells may comprise undifferentiated cells such as hematopoietic stem cells (including bone marrow, circulating and umbilical cells), mesenchymal stem cells, myoblasts (including skeletal and cardiac myoblasts), satellite cells, embryonic stem cells or progenitor cells (including endothelial progenitor cells and cardiac progenitor cells). The cells may also comprise differentiated cells, such as cardiomyocytes, fibroblasts and skeletal myocytes. Such cells can be of embryonic or adult origin and can be obtained from allogeneic, xenogeneic, transgeneic, and autogeneic sources.

In other embodiments, the therapeutic cells are capable of secreting a growth factor or a combination of growth factors, preferably those that are capable of stimulating neovascularization. Examples of suitable growth factors include vascular endothelial growth factor (VEGF), platelet derived growth factor (PDGF-BB, PDGF-CC or PDGF-DD), angiopoietin-1 (Ang-1), acidic fibroblast growth factor (aFGF), basic fibroblast growth factor (bFGF), and transforming growth factor-β1 (TGF-β1) (Carmeliet, Nat. Med. 9:653 (2003)).

The therapeutic cells can provide the therapeutic effect naturally (e.g., cardiomyocytes) or can be recombinantly engineered to provide the effect. For example, the cells can be transformed (i.e., transduced or transfected) with a nucleic acid molecule (preferably DNA) that transforms non-contractile cells in contractile cells or non-secreting cells into secreting cells. Exemplary nucleic acid molecules are those encoding the growth factors described above, as well as MyoD and myogenin (which convert fibroblasts to myocytes). In general, the nucleic acid molecules are operably linked to a suitable genetic control element that is capable of regulating expression of the nucleic acids in a compatible host cell. Suitable genetic control elements include a transcriptional promoter, and may also include transcription enhancers to elevate the level of mRNA expression, a sequence that encodes a suitable ribosome binding site, and sequences that terminate transcription. Suitable eukaryotic promoters include constitutive promoters, as well as inducible promoters of the PolII and PolIII group. In addition, tissue-specific promoters can be used, including cardiac tissue-specific promoters (e.g., the ventricular myosin light chain 2 (MLC-2a and MLC-2v) promoters, sodium-calcium exchanger gene (NCX1) promoters, the slow myosin heavy chain (MyHC3) promoter, the atrial natriuretic factor (ANF) promoter, connexin (CX40, CX43, CX45) promoters, the sacrolipin promoter and the iroquois family homeobox gene (Irx4) promoter) (Small and Krieg, Trends Cardiovasc. Med. 14:13 (2004)).

The cells may be transformed using any appropriate means including viruses (e.g., retrovirus, adenovirus, adeno-associated virus, alphavirus, and lentivirus), chemical transfectants (e.g., cationic polymers, PEI-based transfectants, PLL-based transfectants, dendrimers, polysaccharide-oligoamine based transfectants and cationic lipids), or physio-mechanical methods (e.g., electroporation, microinjection and bioballistics).

In addition to therapeutic cells, the compositions of the present invention can further comprise one or more therapeutic agents in contact with the biocompatible matrix. A wide variety of therapeutic agents can be used in accordance with the present invention. Growth factors such as those described above are among the therapeutic agents preferred for use with the present invention. These growth factors can be delivered as proteins or as nucleic acid molecules encoding them as described above, either alone or in conjunction with an agent that facilitates cellular uptake of biological materials, such as, e.g, viral vectors, cationic lipids, cationic polymers, dendrimers, liposomes and targeting ligands. Angiogenic substances such as, e.g., estrogen, including 17-β estradiol (E2) and estriol (E3), are also believed suitable for use with the present invention. Stabilizing agents, such as, e.g., heparin sulphates and oligomeric regenerating agents (RGTAs), can also be used as the additional therapeutic agent. Potentiating agents, such as for example, nitrous oxide or a nitrous oxide donor, which potentiates the therapeutic effect of VEGF can also be used as the additional therapeutic agent. Examples of nitrous oxide donors that may be used in the present invention are diethylamine nonoate and sodium nitroprusside.

The compositions and methods of the present invention find particular utility in the treatment of heart disease. As used herein, the terms “treat,” “treating,” “treatment,” and similar terms refer to the administration of a composition or method of the present invention to patients, particularly humans, who are suffering from heart disease for alleviating, suppressing, inhibiting, or otherwise reducing the symptoms of heart disease, including atherosclerosis. The terms “treat,” “treating,” “treatment,” and similar terms also are used herein to refer to the prophylactic administration of a composition or method of the present invention to individuals who may be at risk of, or otherwise wish to avoid, heart disease.

The term “heart disease” refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure.

Accordingly, another aspect of the present invention is directed to a method for treating a patient suffering from heart disease, comprising: a) forming one or more channels within a region of a wall of the patient's heart which includes a myocardial layer; and b) delivering to said region a composition comprising living cells and a biocompatible matrix that forms in situ upon exposure to a physiological condition, wherein said living cells provide a therapeutic effect. In some embodiments, the biocompatible matrix further comprises one or more therapeutic agents, such as those described above.

Various means exist for forming intramyocardial channels in a subject and for delivering compositions of the present invention into such channels. Accordingly, another aspect of the present invention is directed to a system for delivering therapeutic cells to the heart of a subject, comprising: a) means for forming one or more channels within a region of a wall of the subject's heart which includes a myocardial layer; (b) means for introducing into said region a composition comprising living cells and a biocompatible matrix that forms in situ upon exposure to a physiological condition, wherein said living cells provide a therapeutic effect.

Such systems find particular utility in the treatment of heart disease. Accordingly, another aspect of the present invention is directed to a system for delivering therapeutic cells to the heart of a patient suffering from heart disease, comprising: a) means for forming one or more channels within a region of a wall of the patient's heart which includes a myocardial layer; (b) means for introducing into said region a composition comprising living cells and a biocompatible matrix that forms in situ upon exposure to a physiological condition, wherein said living cells provide a therapeutic effect.

Means for forming intramyocardial channels are well known in the art and include laser TMR, HF current TMR, catheter-based percutaneous laser and HF current myocardial revascularization, and mechanical transmyocardial and percutaneous myocardial revascularization (Slepian, Cur Interv Cardiol Rep 3:218 (2001)). In some embodiments, mechanical transmyocardial and percutaneous myocardial revascularization is performed using a hollow needle to facilitate delivery of the compositions of the present invention immediately following channel formation.

Means for delivering the compositions of the present invention into intramyocardial channels are also well known in the art and include both direct and catheter-based injection means. For direct injection, a small bolus of selected composition can be loaded into a micro-syringe, e.g., a 100 μL Hamilton syringe, and applied directly from the outside of the heart.

Preferably, however, the methods and systems of the present invention comprise a catheter means for delivery of the compositions of the present invention. For example, a catheter can be introduced from the femoral artery and steered into the left ventricle, which can be confirmed by fluoroscopy. Alternatively, the catheter can be steered into the right ventricle. The catheter generally includes an elongated catheter body, suitably an insulative outer sheath which may be made of polyurethane, polytetrafluoroethylene, silicone, or any other acceptable biocompatible polymer, and a standard lumen extending therethrough for the length thereof, which communicates through to a delivery element. The delivery element can be e.g., a hollow needle, a coated delivery surface, a perfusion port(s), a delivery lumen(s), etc. The use of a catheter-based delivery system facilitates composition delivery immediately upon percutaneous myocardial revascularization. In particular, the use of a needle delivery element in conjunction with a catheter-based delivery system allows the operator to perform both mechanical percutaneous myocardial revascularization and composition delivery using a single device.

The catheter may be guided to the indicated location by being passed down a steerable or guidable catheter having an accommodating lumen, for example, as disclosed in U.S. Pat. No. 5,030,204, or by means of a fixed configuration guide catheter, such as illustrated in U.S. Pat. No. 5,104,393. Alternately, the catheter may be advanced to the desired location within the heart by means of a deflectable stylet, as disclosed in PCT Patent Application WO 93/04724, or by a deflectable guide wire, as disclosed in U.S. Pat. No. 5,060,660. In yet another embodiment, a needle delivery element may be retracted within a sheath at the time of guiding the catheter into the subject's heart.

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope and spirit of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A method for delivering therapeutic cells to the heart of a subject, comprising: a) forming one or more channels within a region of a wall of the subject's heart which includes a myocardial layer; and b) delivering to said region a composition comprising living cells and a biocompatible matrix that forms in situ upon exposure to a physiological condition, wherein said living cells provide a therapeutic effect.

2. The method of claim 1, wherein the cells provide the therapeutic effect naturally.

3. The method of claim 1, wherein the cells are recombinantly engineered to provide the therapeutic effect.

4. The method of claim 1, wherein the therapeutic cells secrete a growth factor.

5. The method of claim 4, wherein the growth factor is selected from the group consisting of vascular endothelial growth factor (VEGF), platelet derived growth factor (PDGF-BB, PDGF-CC or PDGF-DD), angiopoietin-1 (Ang-1), acidic fibroblast growth factor (aFGF), basic fibroblast growth factor (bFGF), and transforming growth factor-β1 (TGF-β1).

6. The method of claim 1, wherein the therapeutic cells are contractile cells.

7. The method of claim 6, wherein the therapeutic cells are selected from the group consisting of hematopoietic stem cells (including bone marrow, circulating and umbilical cells), mesenchymal stem cells, myoblasts (including skeletal and cardiac myoblasts), satellite cells, embryonic stem cells or progenitor cells (including endothelial progenitor cells and cardiac progenitor cells), cardiomyocytes, fibroblasts and skeletal myocytes.

8. The method of claim 7, wherein the therapeutic cells are obtained from allogeneic, xenogeneic, transgeneic, or autogeneic sources.

9. The method of claim 1, wherein the physiological condition is selected from the group consisting of temperature, pH, water content and ion concentration.

10. The method of claim 9, wherein the biocompatible matrix is selected from the group consisting of a thermoplastic paste, an in situ crosslinked system, such as a thermoset or an ion-mediated gelating system; an in situ precipitating system with a sol-gel transition induced by solvent removal, temperature or pH; and an organogel.

11. The method of claim 10, wherein the biocompatible matrix comprises components selected from the group consisting of D,L-lactide, glycolide, ε-caprolactone, trimethylene carbonate, dioxanone, ortho esters, poly(ethylene glycol), alginate, sucrose acetate isobutyrate, poly(lactide-co-glycolide), poly(acrylic acid), poly(N-isopropylacrylamide) (PNIPAAM), methylcellulose (MC), MC-grafted PNIPAAM, poly(ethylene glycol)-poly(lactic acid)-poly(ethyleneglycol) triblocks (PEG-PLA-PEG), PEG-PLA diblock copolymers, poly(ethylene oxide)-poly(propylene oxide)-poly(ethylene oxide) (PEO-PPO-PEO) triblocks (Pluronics® or Poloxamer®), capped PEO-PPO-PEO, PEO-poly(L-lactic acid-co-glycolic acid) (PEO-PLLGA), PEO-poly(DL-lactic acid-co-glycolic acid (PEO-PLGA) block and graft copolymers, PEG-PLGA-PEG, PLGA-PEG-PLGA, poly(organophosphazene)s, chitosan-based and silk-elastin polymers, hydroxypropyl-cellulose (Carbopol®), chitosan, peanut oil and waxes.

12. The components of claim 11, wherein the components are modified to facilitate cell adhesion and cell growth.

13. The modifications of claim 12, wherein the modification includes the introduction of RGD-sites.

14. The method of claim 1, wherein the composition further comprises one or more therapeutic agents.

15. The method of claim 14, wherein the therapeutic agent or agents is selected from the group consisting of vascular endothelial growth factor (VEGF), platelet derived growth factor (PDGF-BB, PDGF-CC or PDGF-DD), angiopoietin-1 (Ang-1), acidic fibroblast growth factor (aFGF), basic fibroblast growth factor (bFGF), and transforming growth factor-β1 (TGF-β1), estrogen, heparin sulphates and oligomeric regenerating agents (RGTAs).

16. The method of claim 1, wherein the subject is a patient suffering from heart disease.

17. A system for delivering therapeutic cells to the heart of a subject, comprising: a) means for forming one or more channels within a region of a wall of the subject's heart which includes a myocardial layer; (b) means for introducing into said region a composition comprising living cells and a biocompatible matrix that forms in situ upon exposure to a physiological condition, wherein said living cells provide a therapeutic effect.

18. The system of claim 17, wherein the channel forming means is selected from the group consisting of laser transmyocardial revascularization, high frequency current transmyocardial revascularization, percutaneous laser myocardial revascularization, high frequency current myocardial revascularization, mechanical transmyocardial revascularization and mechanical percutaneous myocardial revascularization.

19. The system of claim 18, wherein the channel forming means comprises a catheter.

20. The system of claim 18, wherein the channel forming means comprises a hollow needle.

21. The system of claim 17, wherein the composition introducing means comprises a catheter.

22. The system of claim 21, wherein the composition introducing means further comprises a delivery element selected from the group consisting of a hollow needle, a coated delivery surface, a perfusion port and a delivery lumen.

23. The system of claim 17, wherein the subject is a patient suffering from heart disease.
